(19) United States
(12) Patent Application Publication
(10) Pub. No.: US 2004/0247683 A1
(43) Pub. Date: Dec. 9, 2004

(54) Biodegradable nanoparticles incorporating highly hydrophilic positively charged drugs

(76) Inventors: Carmen Popescu, Lisle, IL (US); Hayat Onyuksel, Western Springs, IL (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/832,136

(22) Filed: Apr. 26, 2004

Related U.S. Application Data
(60) Provisional application No. 60/467,400, filed on May 2, 2003.

(51) Int. Cl. A61K 9/14

(52) U.S. Cl. 424/486; 514/35; 514/37

(57) ABSTRACT

Nanoparticles of a biodegradable polymer containing a hydrophilic, cationic drug, like streptomycin, and preparations containing the same, are disclosed. Pharmaceutical preparations containing the nanoparticles are administered, preferably orally, to individuals suffering from a disease or condition, and the nanoparticles release the drug, in vivo, to treat the disease or condition.

### CROSS REFERENCE TO RELATED APPLICATION

This application claims the benefit of U.S. provisional patent application Ser. No. 60/467,400, filed May 2, 2003.

### FIELD OF THE INVENTION

The present invention relates to nanoparticle drug compositions, and to the administration of nanoparticle drug compositions to individuals in need thereof. More particularly, the present invention relates to a drug-delivery system comprising biodegradable polymer nanoparticles containing a hydrophilic, positive-charged drug. The nanoparticle drug composition provides an oral drug-delivery system for drugs that previously were not amenable to oral administration.

### BACKGROUND OF THE INVENTION

It is well known that modern-day drugs are very efficacious with respect to treating acute and chronic diseases. However, many drugs are limited in their route of administration. For example, some drugs cannot be administered orally because they are decomposed in the stomach before absorption. Such drugs must be administered by a different route, such as by parenteral administration. Parenteral and other routes of administration are inconvenient and cumbersome for patients to self-administer, and patient compliance often is impaired.

The administration of highly hydrophilic, positively charged, i.e., cationic, drugs has been problematical because such drugs are not readily absorbed by the gastrointestinal (GI) tract. For example, aminoglycosides are highly hydrophilic, cationic drugs, and are not easily absorbed by the GI tract because the lipoid nature of the cell membrane renders the GI tract highly permeable to lipid soluble (i.e., hydrophobic), but not hydrophilic, substances. Hydrophilic drugs, like aminoglycosdes, are unable to overcome such a barrier. In addition, aminoglycosides are a substrate for the multidrug efflux P-glycoprotein (Pgp) at the GI level. Pgp prevents the absorption of its substrates across the apical brush membrane border of the intestine by mediating their active efflux (S. Banerjee et al., Life Sci., 67, 2011 (2000)). Therefore, aminoglycosides are administered parenterally. This route of administration impairs patient compliance, and also creates epidemiological and financial problems in developing countries.

For example, tuberculosis (TB) is one of the most prevalent diseases in the world. Tuberculosis, which is easily transmitted through the air, already infects 1.9 billion people, and takes the lives of about two million people each year. TB also is becoming increasingly resistant to existing drugs. Presently, an urgent need exists for new anti-TB agents that can shorten the treatment regimen for both the active and latent TB forms, and that effectively treat TB caused by multidrug resistant (MDR) strains.

To avoid drug resistance in the treatment of TB, a four-drug regimen, i.e., isoniazid, rifampin, and pyrazinamide (by oral administration) and streptomycin (by injection), is administered to TB patients. Aminoglycosides, such as streptomycin, are important anti-TB agents, but their utility is restricted by the requirement of parenteral administration, which is inconvenient and creates poor patient compliance. In developing countries, parenteral administration creates the additional risk of HIV/TB transmission because disposable syringes often are not available. It also is theorized that poor patient compliance can lead to the development of drug resistance, and it appears that the frequency of streptomycin resistance among anti-TB drugs is surpassed only by isoniazid. An oral aminoglycoside formulation would overcome these problems associated with the treatment of TB and other diseases.

Currently, no technology exists that can effectively deliver aminoglycosides, or other hydrophilic, cationic drugs, by oral administration. The oral administration route is the most preferred route for drug administration, especially for the treatment of chronic diseases having a long duration and requiring a continuous treatment. Therefore, it would be advantageous to develop more efficient and less cumbersome methods of administering a cationic drug to an individual in the treatment of a disease. As set forth in detail hereafter, the present invention is directed to nanoparticle drug compositions, to pharmaceutical preparations containing a nanoparticle drug composition, and to use of a nanoparticle drug composition to treat a disease. The present invention is further directed to improved drug-delivery systems for administering difficult-to-administer drugs, like aminoglycosides and other highly hydrophilic, positively charged drugs.

Polymeric nanoparticles previously were investigated as carriers for oral drug-delivery systems. Research indicated that oral absorption of nanoparticles predominantly takes place at the intestinal lymphatic tissues level.(i.e., Peyer's patches) (A. Hillery, J. Drug Targeting, 2, 151 (1994)). Now it has been found that loading a hydrophilic, cationic drug in biodegradable nanoparticles facilitates drug uptake for lymphatic circulation to the lungs, while avoiding exposure as a Pgp substrate at the GI level.

Because of excellent bioadhesion, biocompatibility, biodegradability, low cost, and ability to open intercellular tight junctions, naturally occurring polymers, like chitosan (CS), have been used as excipients for oral drug-delivery systems (I. M. Lubben et al., Biomaterials, 22, 687 (2000)). A method for chitosan nanoparticle preparation using the ionic interaction between positively charged CS and the negatively charged tripolyphosphate (TPP) anion has been disclosed (P. Calvo et al., J. Appl. Polym. Sci., 63, 125 (1997)). The resulting nanoparticles showed a good drug-loading capacity.

### SUMMARY OF THE INVENTION

The present invention is directed to a drug-delivery system containing a nanoparticle drug composition comprising nanoparticles of a biodegradable polymer incorporating a highly hydrophilic, positively charged drug. The nanoparticle drug composition is incorporated into a pharmaceutical preparation to provide a drug-delivery system of the present invention. The hydrophilic, cationic drug optionally is complexed with a naturally occurring polymer prior to introduction into, and formation of, the biodegradable polymer nanoparticles.

More particularly, the present invention is directed to a drug-delivery system comprising a pharmaceutical preparation incorporating a present nanoparticle drug composition. In accordance with an important aspect of the present invention, the drug is highly hydrophilic and is positively charged. Preferred drugs are the aminoglycosides.

Another aspect of the present invention is to provide a nanoparticle drug composition wherein the biodegradable polymer is a naturally occurring polymer or a synthetic polymer.

Yet another aspect of the present invention is to incorporate the nanoparticle drug composition into a pharmaceutical preparation, wherein the nanoparticle drug composition can be administered to an individual in a liquid or solid form, either orally or parenterally.

Another aspect of the present invention is to provide a pharmaceutical preparation comprising biodegradable nanoparticles containing a cationic drug that can be administered to an individual in a therapeutically effective amount to treat an acute or chronic disease or condition.

Another aspect of the present invention is to provide a pharmaceutical preparation comprising biodegradable nanoparticles containing a cationic drug that remain intact immediately after administration, and that are capable of releasing the hydrophilic, cationic drug in vivo to treat a disease or condition.

Still another aspect of the present invention is to provide a pharmaceutical preparation comprising a nanoparticle drug composition, wherein a hydrophilic, positively charged drug is an aminoglycoside, such as streptomycin (SM), amikacin, kanamycin, gentamycin, neomycin, netilmicin, spectinomicin, or tobramycin.

Another aspect of the present invention is to provide a biodegradable nanoparticle drug composition comprising a complex of a hydrophilic, cationic drug and a naturally occurring polymer, like dextran sulfate.

Yet another aspect of the present invention is to provide a pharmaceutical preparation comprising a nanoparticle drug composition useful in a method of treating TB and diseases and conditions attributed to Pasteurella, Brucella, Hemophilus, Salmonella, Klepsiella, and Shigella bacteria.

One other aspect of the present invention is to provide alternate routes of administration for the safe, easy, and effective delivery of a hydrophilic, cationic drug, especially to provide an oral or systemic route of administration for aminoglycosides and other hydrophilic, cationic drugs.

Yet another aspect of the present invention is to provide a nanoparticle drug composition for parenteral administration to achieve a sustained release of the hydrophilic, cationic drug after bolus injection. This aspect of the invention frees a patient from connection to intravenous (IV) infusion of a drug for extended time periods in the treatment of a disease or condition.

Another aspect of the present invention is to provide a method of treating a disease treatable by a hydrophilic, cationic drug comprising administering to a mammal in need thereof (a) a pharmaceutical preparation comprising a nanoparticle drug composition of the present invention and, optionally, (b) one or more additional drugs useful in the treatment of the disease.

Still another aspect of the present invention is to provide an article of manufacture comprising:

(a) a packaged pharmaceutical preparation comprising a nanoparticle drug composition of the present invention;

(b) an insert providing instructions for the administration of the nanoparticle drug composition to treat a disease; and

(c) a container for (a) and (b). In preferred embodiments, the insert provides for the oral or systemic administration of the nanoparticle drug composition.

These and other aspects and advantages of the present invention will become apparent from the following detailed description of the preferred embodiments.

### DETAILED DESCRIPTION OF THE PREFERRED EMBODIMENTS

The present invention is directed to a novel drug-delivery system which utilizes a nanoparticle drug composition comprising a hydrophilic, cationic drug incorporated into a biodegradable nanoparticle prepared from a naturally occurring or synthetic polymer. The nanoparticle drug composition is incorporated into a pharmaceutical preparation for administration to an individual in need thereof.

The nanoparticle drug composition comprises a hydrophilic, cationic drug, which optionally has been complexed with a high molecular weight, naturally occurring polymer. The drug or drug complex is admixed with a biodegradable polymer, followed by the addition of an inorganic polyanion, like a condensed phosphate, to form the nanoparticles drug composition.

A pharmaceutical preparation containing the nanoparticle drug composition is useful for the oral, parenteral, buccal, sublingual, rectal, vaginal, or urethral delivery of a hydrophilic, cationic drug. The drug can be, for example, but not limited to, a peptide, a protein, an antibacterial, an antifungal, an antineoplastic, an antiprotozoal, an antiarthritic, or an antiinflammatory agent. In a preferred embodiment, the drug is an aminoglycoside. In especially preferred embodiments, the drug is streptomycin.

The following discussion is particularly directed to the preparation, characterization, and evaluation of a nanoparticle drug composition containing streptomycin (as the drug) and chitosan (as the biodegradable polymer). However, the present invention is. not limited to streptomycin and chitosan. Persons skilled in the art are aware that other cationic drugs having the structural characteristics of streptomycin, especially other aminoglycosides, also can be used as a drug in the nanoparticle drug composition.

In preferred embodiments, a nanoparticle drug composition is prepared from a complex formed between the drug and a naturally occurring polymer. The drug, complexed or uncomplexed, is admixed with the biodegradable polymer followed by the addition of an inorganic polyanion, like a condensed phosphate, to form the nanoparticle drug composition. A pharmaceutical preparation containing the nanoparticle drug composition then can be administered to an individual in need thereof by a variety of routes, including oral and parenteral.

In accordance with an important feature of the present invention, a hydrophilic, cationic drug, like streptomycin, and many other drugs, can be administered orally. Previously, cationic drugs could not be administered orally because such drugs are not absorbed by the GI tract sufficiently to perform their intended function.

The drug present in the nanoparticle drug composition can be any drug that is hydrophilic and has a positive charge. The drug has at least one positively charged site. The positively charged site typically is an ammonium or a quaternary ammonium nitrogen atom. The drug can be a naturally occurring or synthetic drug. The drug can be monomeric, oligomeric, or polymeric, such a polypeptide or protein. Preferred drugs are the aminoglycosides.

If the drug is a synthetic drug, the drug typically contains a nitrogen atom that can be protonated or quaternized. If the drug is a naturally occurring drug, the drug typically contains an amino acid having a positively charged site.

For example, if the drug is insulin, the insulin molecule contains the amino acids lysine, arginine, and histidine. Each of these amino acids has a positively charged site. Similarly, human growth hormone contains 191 amino acids in two polypeptide chains. Human growth hormone also contains the amino acids lysine, arginine, and histidine, which, like insulin, contain positively charged sites.

Other drugs that can be used in the nanoparticle drug composition include, but are not limited to, antiinflammatory drugs, like tereofenamate, proglumetacin, tiaramide, apazone, benzpiperylon, pipebuzone, ramifenazone, and methotrexate; antiinfective drugs, like isoniazid, polymyxin, bacitracin, tuberactionomycin, and erythromycin; antiarthritis drugs, like penicillamine, chloroquine phosphate, glucosamine, and hydroxychloroquine; diabetes drugs, like insulin and glucagons; and anticancer drugs, like cyclophosphamide, interferon α, interferon β, interferon γ, vincristine, and vinblastine.

The naturally occurring polymer optionally used to complex with the drug has a high molecular weight, e.g., a weight average molecular weight (M_{w}) of 25,000 or greater. In general, the naturally occurring polymer has an M_{w} of about 50,000 to about 1,000,000, and preferably about 75,000 to about 750,000. To achieve the full advantage of the present invention, the naturally occurring polymer has an M_{w} of about 100,000 to about 700,000.

Suitable naturally occurring polymers, therefore, include, but are not limited to, dermatan sulfate, chondroitin sulfate, keratin sulfate, heparin sulfate, dextran sulfate, and mixtures thereof. A preferred naturally occurring polymer is dextran sulfate.

The biodegradable polymer used to form the nanoparticles typically is chitosan. However, other naturally occurring and synthetic biodegradable polymers having a cationic character also can be used to form the nanoparticles. Such polymers typically contain a protonated nitrogen atom and are naturally occurring. Examples of other biodegradable polymers include, but are not limited to, collagen, albumin, cellulose, gelatin, elastin, and hyalauronic acid.

To illustrate the present invention, a nanoparticle drug composition containing streptomycin as the drug and chitosan as the biodegradable polymer was prepared. The nanoparticle drug composition is useful for the oral administration of streptomycin or the sustained release of streptomycin after parenteral administration.

The nanoparticle drug composition was prepared in general as follows:

(a) the positive charge of streptomycin was partially neutralized by the addition of a naturally occurring polymer (e.g., dextran sulfate), which formed a drug complex;

(b) the drug complex was added to an aqueous solution the biodegradable polymer (e.g., chitosan); then

(c) a polyphosphate was added to the product of (b) to form the chitosan nanoparticles incorporating the streptomycin drug complex. The nanoparticle drug composition had a particle size range of about 50 to about 500 nm.

In particular, a novel oral delivery system containing streptomycin (SM) in biodegradable chitosan nanoparticles was prepared and tested for in vivo efficacy using an M. tuberculosis (TB) chronic infection mouse model. Test results show that the SM-chitosan nanoparticles, administered orally, were as effective as a subcutaneously injected, aqueous SM solution. The method of Janes et al., J. Contr. Rel., 73, 255 (2001), incorporated herein by reference, was used to entrap a cationic, hydrophilic drug, such as SM, into chitosan nanoparticles, i.e., complexation of SM with dextran sulfate (a polyanion) followed by chitosan nanoparticle preparation using the conventional tripolyphosphate (TPP) method.

The in vitro physicochemical properties of the nanoparticle drug composition, and the in vivo efficacy of the SM-chitosan nanoparticles, after oral administration for three weeks in an M. tuberculosis chronic infection mouse model, was determined.

Experimental Methods

Preparation of the SM Chitosan Nanoparticles

Chitosan (0.2% w/v) was dissolved in aqueous acetic acid solution (0.1N). Then, 20 ml of an SM solution (0.2% w/v) was incubated with 20 ml dextran sulfate (MW 500,000) (0.15% w/v) for 30 seconds. The resulting complex was added to 80 ml of a chitosan solution. The addition of 20 ml TPP solution (0.08% w/v) with stirring led to the immediate formation of SM-chitosan nanoparticles.

Characterization of the SM Chitosan Nanoparticles

Particle size and zeta potential of the nanoparticles were measured by quasielastic light scattering NICOMP (Model 380) and by Lazer Zee Meter (Model 501). For size measurement, samples were diluted in water and measured for 30 min. For zeta potential measurement the samples were diluted with a 0.1 mM KCl solution.

SM encapsulation was determined by ultracentrifuge sedimentation at 40,000 g (15° C.) for 30 min using a Beckman ultracentrifuge (Optima™ LE-80K). The unencapsulated SM concentration in the supernatant was determined using a spectrophotometric method as described in S. E. Katz, J. Agric. Food Chem., 8, 501 (1960). The SM incorporation efficiency was calculated as described in K. A. Janes et al. All measurements were performed in triplicate.

Mouse Infection Model and Treatment

The SM chitosan nanoparticles were concentrated by ultracentrifugation at 10,000 g for 30 min, followed by resuspension of the nanoparticles in distilled water. The SM final concentration was 20 mg/ml.

BALB/c mice (about 20 g) were infected by aerosol with M. tuberculosis Erdman. See S. L. Baldwin et al., Infect. Immun., 66(6), 2951 (1998). Beginning at 45 days post infection, the mice were treated daily for 3 weeks at 100 mg/kg either with SM loaded chitosan nanoparticles by oral gavage or injected subcutaneously with SM solution (in water). Untreated mice were used as controls. At the end of the treatment, colony-forming units (CFU) in the lungs were counted for each group. The statistical significance of all results was determined using the two-tailed Student's t-test.

The mean size and zeta potential values of the SM-chitosan nanoparticles were 557.93±100.38 nm and +52.07±3.4 mV, respectively. Drug incorporation efficiency of SM in the chitosan nanoparticles was 52.11±0.71%. This is an unexpectedly high incorporation efficiency value because SM is positively charged, and chitosan also is a positively charged polysaccharide in acetic acid solution, which was expected to cause problems during SM-chitosan nanoparticle formation. Accordingly, dextran sulfate (M_{w} 500,000) was used to decrease the cationic character of SM. It was found that using a low M_{w} dextran sulfate (e.g., M_{w} 10,000) lowered the incorporation efficiency of SM into the chitosan nanoparticles to 21.66%.

Surprisingly, it also was found that a one log₁₀ reduction (p<0.01) in growth of the TB bacilli was achieved for both treated groups (i.e., oral SM-chitosan nanoparticles and injected SM) compared to the control group. In particular, mice in the control test had a log CFU in the lungs of 6.88. The SM-chitosan nanoparticle-treated group had a reduced log CFU of 5.91. The injected CM treated group had a log CFU of 6.13. This test was repeated using oral SM dosages of 200 mg/kg and 400 mg/kg. The log CFU for the SM-chitosan nanoparticles treated mice in these tests was 6.35 and 6.15, respectively (control log CFU 6.88). These results show that orally administered SM-chitosan nanoparticles were as effective in killing intracellular M. tuberculosis as subcutaneously injected SM (p>0.05).

In the development of tuberculosis therapy, it is important that the tubercle bacilli are facultative intracellular parasites, especially in the chronic phase of the disease (E. L. W. Barrow et al., Antimicroagents and Chemotherapy, 42, 2682 (1998)). Although it is known that SM is highly bactericidal against rapidly dividing M. tuberculosis, SM has less activity against bacilli that are not multiplying and are in intracellular (J. Dhillon et al., J. Antimicrob. Chemother., 48, 869 (2001)), as in the chronic infection model used in this study.

A hypothesis for this relatively low activity may be poor penetration and retention of SM within the host cells, and reduced activity of SM in the acidic cell environment (pH5.0) (P. Couvreur et al., Pharm. Res., 8, 1079 (1991)). Therefore, the unexpectedly high efficacy of the present SM-chitosan nanoparticles may be explained by several unrelied upon mechanisms. For example, chitosan nanoparticles may have enhanced the drug permeability through the tight junctions, or/and SM-chitosan nanoparticles may have been taken up by the M. tuberculosis cells and delivered to the lungs through lymphatic circulation. After being phagocytized by macrophages, the nanoparticles can deliver the SM exactly where the tubercle bacilli reside. Under either hypothesis, Pgp-mediated efflux is avoided. Furthermore, the SM-chitosan nanoparticles also may protect the drug from the acid environment in the cell. It is hypothesized, therefore, but not relied upon, that these combined factors contribute to the high efficacy of orally administered SM-chitosan nanoparticles.

Streptomycin is not orally bioavailable and its oral delivery would greatly facilitate its use in the treatment of tuberculoses and other diseases. The present nanoparticle drug composition permits the oral delivery of streptomycin. However, the nanoparticle drug composition also can be administered by other routes of administration.

For example, the nanoparticle drug composition can be formulated in suitable excipients for oral administration or for parenteral administration. Such excipients are well known in the art. The nanoparticle drug composition typically is present in such a pharmaceutical preparation in an amount of about 0.1% to about 75% by weight.

Pharmaceutical preparations containing a nanoparticle drug composition of the present invention are suitable for administration to humans or other mammals. Typically, the pharmaceutical preparations are sterile, and contain no toxic, carcinorgenic, or mutagenic compound which would cause an adverse reaction when administered.

The nanoparticle drug composition can be administered by any suitable route, for example by oral, buccal, inhalation, sublingual, rectal, vaginal, intracisternal through lumbar puncture, transurethral, nasal, or parenteral (including intravenous, intramuscular, subcutaneous, and intracoronary) administration. Parenteral administration can be accomplished using a needle and syringe. Implant pellets also can be used to administer a nanoparticle drug composition parenterally. The nanoparticle drug composition also can be administered as a component of an ophthalmic drug-delivery system.

The pharmaceutical preparations include those wherein the nanoparticle drug composition is administered in an effective amount to achieve its intended purpose. More specifically, a “therapeutically effective amount” means an amount effective to treat a disease. Determination of a therapeutically effective amount is well within the capability of those skilled in the art, especially in light of the detailed disclosure provided herein.

The exact formulation, route of administration, and dosage is determined by an individual physician in view of the patient's condition. Dosage amount and interval can be adjusted individually to provide levels of the nanoparticle drug composition that are sufficient to maintain therapeutic or prophylactic effects.

The amount of pharmaceutical preparation administered is dependent on the subject being. treated, on the subject's weight, the severity of the affliction, the manner of administration, and the judgment of the prescribing physician.

Specifically, for administration to a human in the curative or prophylactic treatment of a disease, oral dosages of the nanoparticle drug composition is about 10 to about 500 mg daily for an average adult patient (70 kg). Thus, for a typical adult patient, individual doses contain about 0.1 to about 500 mg nanoparticle drug composition, in a suitable pharmaceutically acceptable vehicle or carrier, for administration in single or multiple doses, once or several times per day. Dosages for intravenous, buccal, or sublingual administration typically are about 0.1 to about 10 mg/kg per single dose as required. In practice, the physician determines the actual dosing regimen that is most suitable for an individual patient and disease, and the dosage varies with the age, weight, and response of the particular patient. The above dosages are exemplary of the average case, but there can be individual instances in which higher or lower dosages are merited, and such are within the scope of this invention.

A nanoparticle drug composition of the present invention can be administered alone, or in admixture with a pharmaceutical carrier selected with regard to the intended route of administration and standard pharmaceutical practice. Pharmaceutical preparations for use in accordance with the present invention, including ophthalmic preparations, thus can be formulated in a conventional manner using one or more physiologically acceptable carriers comprising excipients and auxiliaries that facilitate processing of a nanoparticle drug composition into preparations that can be used pharmaceutically.

These pharmaceutical preparations can be manufactured in a conventional manner, e.g., by conventional mixing, dissolving, granulating, dragee-making, emulsifying, or lyophilizing processes. Proper formulation is dependent upon the route of administration chosen. When a therapeutically effective amount of the nanoparticle drug composition is administered orally, the formulation typically is in the form of a tablet, capsule, powder, solution, or elixir. When administered in tablet form, the composition additionally can contain a solid carrier, such as a gelatin or an adjuvant. The tablet, capsule, and powder contain about 5% to about 95%, preferably about 25% to about 90%, of a nanoparticle drug composition of the present invention. When administered in liquid form, a liquid carrier, such as water, petroleum, or oils of animal or plant origin, can be added. The liquid form of the pharmaceutical preparation can further contain physiological saline solution, dextrose or other saccharide solutions, or glycols. When administered in liquid form, the pharmaceutical preparation contains about 0.5% to about 90%, by weight, of a nanoparticle drug composition, and preferably about 1% to about 50%, by weight, of a nanoparticle drug composition.

When a therapeutically effective amount of a nanoparticle drug composition is administered by intravenous, cutaneous, or subcutaneous injection, the composition is in the form of a pyrogen-free, parenterally acceptable aqueous preparation. The preparation of such parenterally acceptable solutions, having due regard to pH, isotonicity, stability, and the like, is within the skill in the art. A preferred preparation for intravenous, cutaneous, or subcutaneous injection typically contains an isotonic vehicle in addition to a nanoparticle drug composition of the present invention.

A nanoparticle drug composition can be readily combined with pharmaceutically acceptable carriers well-known in the art. Such carriers enable the nanoparticle drug composition to be formulated as tablets, pills, dragees, capsules, liquids, gels, syrups, slurries, suspensions and the like, for oral ingestion by a patient to be treated. Pharmaceutical preparations for oral use can be obtained by adding the nanoparticle drug composition with a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

A nanoparticle drug composition can be formulated for parenteral administration by injection, e.g., by bolus injection or continuous infusion. Preparations for injection can be presented in unit dosage form, e.g., in ampules or in multidose containers, with an added preservative. The preparations can take such forms as suspensions, solutions, or emulsions in oily or aqueous vehicles, and can contain formulatory agents such as suspending, stabilizing, and/or dispersing agents.

Pharmaceutical preparations for parenteral administration include aqueous dispersions of the nanoparticle drug composition. Additionally, suspensions of the nanoparticle drug composition can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the dispersibility of the compounds and allow for the preparation of highly concentrated preparations. Alternatively, a present pharmaceutical preparation can be in powder form for constitution with a suitable vehicle, e.g., sterile pyrogen-free water, before use.

A nanoparticle drug composition also can be formulated in rectal compositions, such as suppositories or retention enemas, e.g., containing conventional suppository bases. In addition to the preparations described previously, the nanoparticle drug composition also can be formulated as a depot preparation. Such long-acting preparations can be administered by implantation (for example, subcutaneously or intramuscularly) or by intramuscular injection. Thus, for example, the nanoparticle drug composition can be formulated with suitable polymeric or hydrophobic materials (for example, as an emulsion in an acceptable oil) or ion exchange resins.

In particular, the nanoparticle drug composition can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. A formulation also can be injected parenterally, for example, intravenously, intramuscularly, subcutaneously, or intracoronarily. For parenteral administration, the formulation is best used in the form of a sterile aqueous solution which can contain other substances, for example, salts, or monosaccharides, such as mannitol or glucose, to make the solution isotonic with blood.

For veterinary use, the nanoparticle drug composition is administered as a suitably acceptable formulation in accordance with normal veterinary practice. The veterinarian can readily determine the dosing regimen and route of administration that is most appropriate for a particular animal.

The present invention, therefore, discloses a novel drug-delivery system for the oral, parenteral, sublingual, rectal, vaginal, or urethral delivery of therapeutic agents. The drug-delivery system is a pharmaceutical preparation comprising nanoparticles comprising a hydrophilic, positively charged drug, optionally in complexed form, and a biodegradable polymer. The drug, or drug complex, is entrapped in a nanoparticle of the biodegradable polymer. The pharmaceutical preparations then can be administered by a variety of oral and parenteral routes.

In addition, although the present disclosure is particularly directed to the preparation of a streptomycin-loaded chitosan nanoparticle, persons skilled in the art can apply this technology to a variety of drugs and nanoparticle-forming, biodegradable polymers.

As demonstrated herein, streptomycin was successfully loaded in chitosan nanoparticles with high incorporation efficiency of 50% or higher, and a loading efficiency of 30% or higher. The nanoparticles also can contain other aminoglycosides (e.g., amikacin, gentamycin, tobramycin, kanamycin, and neomycin) because they have similar physiochemical properties to streptomycin. The streptomycin chitosan nanoparticles were orally bioavailable and as effective in killing intracellular M. tuberculosis as subcutaneously injected streptomycin solution.

Modifications and variations of the invention as hereinbefore set forth can be made without departing from the spirit and scope thereof, and only such limitations should be imposed as are indicated by the appended claims.

What is claimed is:

1. A composition comprising:
(a) an aminoglycoside; and
(b) a naturally occurring polymer,
wherein the composition comprises nanoparticles having mean particle size of about 1 nm to about 1000 nm.

2. The composition of claim 1 wherein the aminoglycoside comprises streptomycin, amikacin, kanamycin, gentamicin, neomycin, netilmicin, spectinomycin, or tobramycin.

3. The composition of claim 1 wherein the mean particle size is about 50 nm to about 500 nm.

4. The composition of claim 1 further comprising a polyanionic salt.

5. The composition of claim 4 wherein the polyanionic salt comprises a condensed polyphosphate.

6. The composition of claim 5 wherein the condensed polyphosphate comprises, a diphosphate, a triphosphate, or a derivative thereof.

7. The composition of claim 5 wherein the polymer is ionically associated with the condensed polyphosphate.

8. The composition of claim 1 wherein the polymer is capable of ionically associating with a polyanionic salt.

9. The composition of claim 1 wherein the polymer comprises a protein.

10. The composition of claim 1 wherein the polymer comprises a polysaccharide.

11. The composition of claim 1 wherein the polymer comprises a nitrogen atom.

12. The composition of claim 1 wherein the polymer is protonated.

13. The composition of claim 1 wherein the polymer has a molecular weight of 25,000 g/mol or greater.

14. The composition of claim 1 wherein the polymer has a molecular weight about 100,000 g/mol to about 700,000 g/mol.

15. The composition of claim 1 wherein the polymer comprises chitosan, dextran sulfate, dermatan sulfate, chondroitin sulfate, keratin sulfate, heparin sulfate, collagen, albumen, cellulose, gelatin, elastin, hyalauronic acid, or mixtures thereof.

16. The composition of claim 1 wherein the polymer comprises chitosan and the aminoglycoside comprises streptomycin.

17. The composition of claim 1 in oral dosage form.

18. A method of treating a disease or medical condition in a mammal comprising administering to a mammal in need of such treatment a therapeutically effective amount of a composition comprising:
(a) an aminoglycoside; and
(b) a naturally occurring polymer,
wherein the composition comprises nanoparticles having mean particle size of about 1 nm to about 1000 nm.

19. The method of claim 18 wherein the aminoglycoside comprises streptomycin, amikacin, kanamycin, gentamicin, neomycin, netilmicin, spectinomycin, or tobramycin.

20. The method of claim 18 wherein the mean particle size is about 50 nm to about 500 nm.

21. The method of claim 18 wherein the composition further comprises a polyanionic salt.

22. The method of claim 21 wherein the polyanionic salt comprises a condensed polyphosphate.

23. The method of claim 22 wherein the condensed polyphosphate comprises a diphosphate, a triphosphate, or a derivative thereof.

24. The method of claim 22 wherein the polymer is ionically associated with the condensed polyphosphate.

25. The method of claim 18 wherein the polymer is capable of ionically associating with a polyanionic salt.

26. The method of claim 18 wherein the polymer comprises a polysaccharide.

27. The method of claim 18 wherein the polymer comprises a nitrogen atom.

28. The method of claim 18 wherein the polymer comprises a protein.

29. The method of claim 18 wherein the polymer is protonated.

30. The method of claim 18 wherein the polymer has a molecular weight of 25,000 g/mol or greater.

31. The method of claim 18 wherein the polymer has a molecular weight of about 100,000 g/mol to about 700,000 g/mol.

32. The method of claim 18 wherein the polymer comprises chitosan, dextran sulfate, dermatan sulfate, chondroitin sulfate, keratin sulfate, heparin sulfate, collagen, albumen, cellulose, gelatin, elastin, hyalauronic acid, or mixtures thereof.

33. The method of claim 18 wherein the polymer comprises chitosan and the aminoglycoside compound comprises streptomycin.

34. The method of claim 18 wherein the composition is administered orally.

35. A pharmaceutical formulation for treating a disease or condition in a mammal comprising:
(a) a composition comprising:
(i) a therapeutically effective amount of a bioactive compound, and
(ii) either:
(1) a naturally occurring polymer capable of ionically associating with a condensed polyphosphate salt, or
(2) a polysaccharide, wherein the composition comprises nanoparticles of a mean particle size of from about 1 nm to about 1000 nm; and
(b) a pharmaceutically acceptable carrier.

36. The formulation of claim 35 wherein the composition further comprises the condensed polyphosphate.

37. The formulation of claim 36 wherein the condensed polyphosphate comprises a diphosphate, or a triphosphate, or a derivative thereof.

38. The formulation of claim 36 wherein the polymer or the polysaccharide is ionically associated with the condensed polyphosphate.

39. The formulation of claim 35 wherein the mean particle size of about 50 nm to about 500 nm.

40. The formulation of claim 35 wherein the bioactive compound is a salt.

41. The formulation of claim 35 wherein the bioactive compound comprises a nitrogen atom.

42. The formulation of claim 35 wherein the bioactive compound is a substrate for p-glycoprotein.

43. The formulation of claim 35 wherein the bioactive compound comprises an aminogylcoside, a polypeptide, a protein, insulin, human growth hormone, tereofenamate, proglumetacin, tiaramide, apazone, benzpiperylon, pipebuzone, ramifenazone, methotrexate, isoniazid, polymyxin, bacitracin, tuberactionomycin, ethryomycin, penicillamine, chloroquine phosphate, glucosamine, hydroxychloroquine, glucagons, cyclophosphamide, interferon α, interferon β, interferon γ, vincristine, or vinblastine.

44. The formulation of claim 43 wherein the aminoglycoside comprises streptomycin, amikacin, kanamycin, gentamicin, neomycin, netilmicin, spectinomycin, or tobramycin.

45. The formulation of claim 35 wherein the polymer comprises a protein.

46. The formulation of claim 35 wherein the polymer or the polysaccharide comprises a nitrogen atom.

47. The formulation of claim 35 wherein the polymer or the polysaccharide is protonated.

48. The formulation of claim 35 wherein the polymer or the polysaccharide has a molecular weight of 25,000 g/mol or greater.

49. The formulation of claim 35 wherein the polymer or the polysaccharide has a molecular weight of about 100,000 g/ mol to about 700,000 g/mol.

50. The formulation of claim 35 wherein the polymer or the polysaccharide comprises chitosan, dextran sulfate, dermatan sulfate, chondroitin sulfate, keratin sulfate, heparin sulfate, collagen, albumen, cellulose, gelatin, elastin, or hyalauronic acid, or mixtures thereof.

51. The formulation of claim 35 wherein the polymer or the polysaccharide comprises chitosan and the bioactive compound comprises streptomycin.

52. The formulation of claim 35 in oral dosage form.

53. A method of treating a disease or medical condition in a mammal comprising administering to a mammal in need of such treatment a therapeutically effective amount of a composition comprising:
(a) a bioactive compound; and
(b) either:
(i) a naturally occurring polymer capable of ionically associating with a polyphosphate salt, or
(ii) a polysaccharide,
wherein the composition comprises nanoparticles of mean particle size of from about 1 nm to about 1000 nm.

54. The method of claim 53 wherein the composition further comprises the condensed polyphosphate.

55. The method of claim 54 wherein the condensed polyphosphate comprises a diphosphate, or a triphosphate, or a derivative thereof.

56. The method of claim 54 wherein the polymer or the polysaccharide is tonically associated with the polyphosphate salt.

57. The method of claim 53 wherein the mammal is a human.

58. The method of claim 53 comprising orally administering the composition.

59. The method of claim 53 wherein the disease or medical condition comprises a bacterial infection.

60. The method of claim 53 wherein the disease or medical condition is tuberculosis.

61. The method of claim 53 wherein the composition further comprises a pharmaceutically acceptable carrier.

62. The method of claim 53 wherein the mean particle size of the nanoparticles is about 50 nm to about 500 nm.

63. The method of claim 53 wherein the bioactive compound is a salt.

64. The method of claim 53 wherein the bioactive compound comprises a nitrogen atom.

65. The method of claim 53 wherein the bioactive compound is a substrate for p-glycoprotein.

66. The method of claim 53 wherein the bioactive compound comprises an aminoglycoside, a polypeptide, a protein, insulin, human growth hormone, tereofenamate, proglumetacin, tiaramide, apazone, benzpiperylon, pipebuzone, ramifenazone, methotrexate, isoniazid, polymyxin, bacitracin, tuberactionomycin, erythromycin, penicillamine, chloroquine phosphate, glucosamine, hydroxychloroquine, glucagons, cyclophosphamide, interferon α, interferon β, interferon γ, vincristine, or vinblastine.

67. The method of claim 53 wherein the bioactive compound comprises an aminoglycoside.

68. The method of claim 67 wherein the aminoglycoside comprises streptomycin, amikacin, kanamycin, gentamicin, neomycin, netilmicin, spectinomycin, or tobramycin.

69. The method of claim 53 wherein the polymer comprises a protein.

70. The method of claim 53 wherein the polymer or the polysaccharide comprises a nitrogen atom.

71. The method of claim 53 wherein the polymer or the polysaccharide is protonated.

72. The method of claim 53 wherein the polymer or the polysaccharide has a molecular weight of 25,000 g/mol or greater.

73. The method of claim 53 wherein the polymer or the polysaccharide has a molecular weight of from about 100,000 g/ mol to about 700,000 g/mol.

74. The method of claim 53 wherein the polymer or the polysaccharide comprises chitosan, dextran sulfate, dermatan sulfate, chondroitin sulfate, keratin sulfate, heparin sulfate, collagen, albumen, cellulose, gelatin, elastin, hyalauronic acid, or mixtures thereof.

75. The method of claim 53 wherein the polymer or the polysaccharide comprises chitosan and the bioactive compound comprises streptomycin.

76. The method of claim 53 wherein the composition is in oral dosage form.

77. A method of treating tuberculoses comprising orally administering to a mammal in need of such treatment a therapeutically effective amount of an aminoglycoside.

78. The method of claim 77 wherein the aminoglycoside comprises streptomycin, amikacin, kanamycin, gentarnicin, neomycin, netilmicin, spectinomycin, or tobramycin.

79. A drug-delivery system comprising a nanoparticle drug composition, said composition comprising:
(a) a hydrophilic, cationic drug incorporated into
(b) nanoparticles of a biodegradable polymer.

80. The system of claim 1 wherein the aminoglycoside is selected from the group consisting of streptomycin, kanamycin, neomycin, gentamycin, amikacin, netilmicin, spectinomycin, and tobramycin.

81. A method of treating a disease or condition treatable by an aminoglycoside comprising administering a therapeutically effective amount of a drug-delivery system of claim 79, to an individual in need thereof, wherein the drug comprises an aminoglycoside.

82. The method of claim 81 wherein the drug-delivery system is administered orally.

83. The method of claim 82 wherein the drug-delivery system is administered parenterally.
